(19) United States
(12) Patent Application Publication
(10) Pub. No.: US 2026/0001797 A1
(43) Pub. Date: Jan. 1, 2026

(54) ALGAE CULTIVATION AND HARVESTING FOR NUTRIENT REMEDIATION

(71) Applicant: AECOM, Los Angeles, CA (US)

(72) Inventor: Daniel J. Levy, Fort Lauderdale, FL (US)

(21) Appl. No.: 19/251,241

(22) Filed: Jun. 26, 2025

Related U.S. Application Data
(60) Provisional application No. 63/666,069, filed on Jun. 28, 2024.

(51) Int. Cl. C02F 3/32; A01G 33/00; C02F 101/10; C02F 101/16; C02F 103/00; C12N 1/12

(52) U.S. Cl. CPC C02F 3/322; A01G 33/00; C12N 1/12; C02F 2101/105; C02F 2101/16; C02F 2103/007

(57) ABSTRACT

A method for surface water remediation and prevention and mitigation of harmful algal blooms includes the steps of receiving nutrient-rich water from a body of water; cultivating a first type of algae in the raceway algae growth pond to absorb and reduce nutrient concentrations in the nutrient-rich surface water; flowing at least a portion of the nutrient-rich surface water treated in the raceway algae growth pond to an attached algae flow way connected downstream of the raceway algae growth pond; cultivating a second type of algae in the attached algae flow way to further absorb and reduce nutrient concentrations in the nutrient-rich surface water; and returning at least a portion of the nutrient-rich surface water treated by the raceway algae growth pond and the attached algae flow way to the body of water.

### CROSS-REFERENCE TO RELATED APPLICATIONS

This application claims the benefit of U.S. Provisional Application No. 63/666,069, entitled “ALGAE CULTIVATION AND HARVESTING FOR NUTRIENT REMEDIATION” and filed on Jun. 28, 2024, which is hereby incorporated by reference in its entirety.

### TECHNICAL FIELD

The present invention relates generally to systems and methods for algae cultivation and harvesting for nutrient remediation in bodies of water.

### BACKGROUND

A key issue limiting the prevention of harmful algal blooms (HABs) in bodies of water such as rivers lakes, streams, etc., is the contribution of nutrient loads from headwater streams to downstream water bodies. Many of these streams have elevated dissolved nitrogen (N) and phosphorus (P) concentrations due to non-point sources in the catchments including large stores of legacy nutrients that have accumulated in soils over many decades. These nutrients continue to be released to headwater streams via diffuse groundwater and surface water runoff even after restoration and best management practices are implemented to prevent further supply of nutrients to the water. Reducing the supply these nutrient loads to headwater streams is especially challenging for those that drain large areas of historic contamination such as areas of former phosphate mining and septic system usage. There is a need for new technologies that can address the legacy nutrients in bodies of water.

### SUMMARY

The techniques described herein relate to a method for surface water remediation and prevention and mitigation of harmful algal blooms. The method includes providing water from a surface water source to a raceway pond; cultivating algae in the raceway pond to absorb and reduce nutrient concentrations; and providing water from the raceway pond to an algae flow way connected downstream of the raceway pond; and cultivating algae in the algae flow way to further reduce nutrient concentrations in the water.

In some aspects, the raceway pond and the algae flow way can be operated as side-stream process for treating water from the surface water source and returning water to the surface water source. The method can reduce nutrient concentrations in the surface water source to less than about 0.1 mg/L. In some cases, the method can include harvesting algae cultivated in the raceway pond and the algae flow way to produce a commercial biofertilizer. In some aspects, the method includes removing algae from the algae flow way to enhance nutrient removal efficiency. Cultivating algae in the raceway pond can reduce a total phosphorus concentration in the water from more than about 4 mg/L to less than about 0.5 mg/L. In some cases, cultivating algae in the algae flow way can reduce the total phosphorus concentration to less than about 0.005 mg/L. In some aspects, cultivating algae in the raceway pond can reduce a total nitrogen concentration in the water from more than about 19 mg/L to less than about 2 mg/L. Cultivating algae in the algae flow way can reduce the total nitrogen concentration in the water to less than about 0.02 mg/L.

In some cases, the techniques described herein relate to a system for surface water remediation and prevention and mitigation of harmful algal blooms. The system can include a raceway pond for cultivating algae to absorb and reduce nutrient concentrations in nutrient-rich surface water from a main water body; an algae flow way positioned downstream of the raceway pond for supporting the growth of algae to further reduce nutrient concentrations; a water pathway connecting the raceway pond and the algae flow way.

In some aspects, the system includes an algae separation system configured to harvest algae from the raceway pond and/or the algae flow way. The raceway pond can be specifically configured to handle higher nutrient concentrations, and/or the algae flow way can be configured to achieve further nutrient reductions to very low levels.

In some aspects, the techniques described herein relate to a method for surface water remediation, the method including: receiving source water from a body of surface water in a raceway algae growth pond; cultivating a first type of algae in the raceway algae growth pond to absorb and reduce nutrient concentrations in the source water; flowing at least a portion of water treated by the raceway algae growth pond to an attached algae flow way connected downstream of the raceway algae growth pond; cultivating a second type of algae in the attached algae flow way to further absorb and reduce nutrient concentrations in the water treated by the raceway algae growth pond; and returning at least a portion of the water treated by the raceway algae growth pond and the attached algae flow way to the body of surface water.

In some aspects, the raceway algae growth can be is configured to reduce a total phosphorus (TP) concentration of the source water from about 4 mg/L or more to about 0.5 mg/L or less in the water treated by the raceway algae growth pond. The attached algae flow way can be configured to reduce a total phosphorus (TP) concentration of the water treated by the raceway algae growth pond to about 0.005 mg/L or less in the water treated by the attached algae flow way. In some cases, the raceway algae growth pond can be configured to reduce a total nitrogen (TN) concentration of the source water from about 19 mg/L or more to about 2 mg/L or less in the water treated by the raceway algae growth pond. In some aspects, the attached algae flow way can be configured to reduce a total phosphorus (TP) concentration of the water treated by the raceway algae growth pond to about 0.02 mg/L or less in the water treated by the attached algae flow way. The raceway algae growth pond can be configured to reduce the nutrient concentration of the source water by at least about 90%. In some cases, the attached algae flow way can be configured to reduce the nutrient concentration of the water treated by the raceway algae growth pond by at least about 80%.

In some aspects, the first type of algae can include planktonic algae. The second type of algae can include periphytic algae. In some cases, the first type of algae can be the same as the second type of algae. In some aspects, the method can include harvesting the first type of algae from the raceway algae growth pond. The method can include harvesting the second type of algae from the attached algae flow way.

In some aspects, the techniques described herein relate to a system for surface water remediation. The system can include a raceway algae growth pond configured to receive source water from a surface body of water and cultivate a first type of algae to absorb and reduce nutrient concentrations in the source water; and an attached algae flow way positioned downstream of the raceway algae growth pond and configured to receive at least a portion of water treated by the raceway algae growth pond and cultivate a second type of algae to further absorb and reduce nutrient concentrations in the water received from the raceway algae growth pond.

In some aspects, the raceway algae growth pond can be configured to reduce a total phosphorus (TP) concentration of the source water from about 4 mg/L or more to about 0.5 mg/L or less. The attached algae flow way can be configured to reduce a total phosphorus (TP) concentration of the water treated by the raceway algae growth pond to about 0.005 mg/L or less in the water treated by the attached algae flow way. In some cases, the raceway algae growth pond can be configured to reduce a total nitrogen (TN) concentration of the source water from about 19 mg/L or more to about 2 mg/L or less in the water treated by the raceway algae growth pond. In some aspects, the attached algae flow way can be configured to reduce a total phosphorus (TP) concentration of the water treated by the raceway algae growth pond to about 0.02 mg/L or less in the water treated by the attached algae flow way. The raceway algae growth pond can be configured to reduce the nutrient concentration of the source water by at least about 90%. In some cases, the attached algae flow way can be configured to reduce the nutrient concentration of the water treated by the raceway algae growth pond by at least about 80%.

The first type of algae can include planktonic algae. In some cases, the second type of algae can include periphytic algae. In some aspects, the system can include a harvester for harvesting the first type of algae from the raceway algae growth pond. The system can include a harvester for harvesting the second type of algae from the attached algae flow way. In some cases, the system can include a holding tank positioned downstream of the attached algae flow way and configured to receive and store at least a portion of the water treated by the raceway algae growth pond and the attached algae flow way. In some aspects, the system can include flow path from the attached algae flow way to the surface body of water.

### BRIEF DESCRIPTION OF THE DRAWINGS

FIG. 1 shows an example of an algae cultivation and harvesting system.

FIG. 2 shows a schematic diagram of a system for algae cultivation and harvesting.

### DETAILED DESCRIPTION

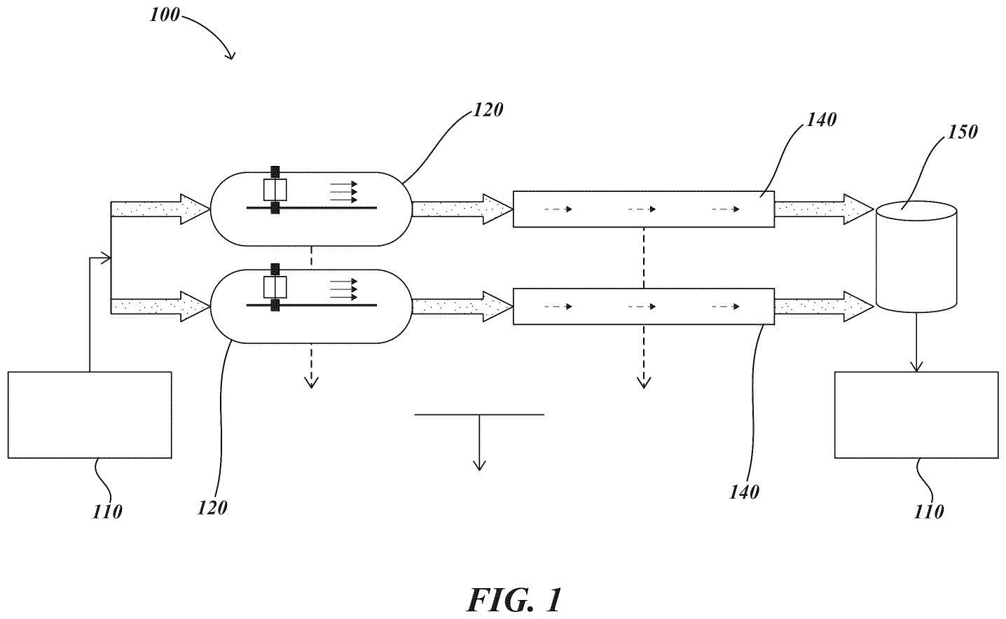

FIG. 1 shows an example of a system 100 for algae cultivation and harvesting. The system 100 receives and returns water to a fresh water source 110, particularly a natural surface fresh water source, such as a river, lake or pond. The system can include one or more raceway ponds 120 and one or more attached algae flow ways 140. As further described below, the one or more raceway ponds 120 and the one or more attached algae flow ways 140 can cultivate and/or harvest the same type or different types of algae. For example, the one or more raceway ponds 120 can cultivate and/or harvest a first type of algae (e.g., planktonic algae) and the one or more attached algae flow ways 140 can cultivate and/or harvest a second type of algae (e.g., periphytic algae biomass).

In some cases, the raceway ponds 120 can receive source water from the fresh water source 110 such as a river, a pond, a stream, etc. In such cases, the raceway ponds can be located close to the water source 110. For example, in some cases, the raceway ponds can receive and treat nutrient-rich surface water. The raceway ponds 120 can cultivate algae to absorb and reduce nutrient concentrations. In some cases, the algae can include planktonic filamentous microalgae. Other examples of algae include, but are not limited to, Uronema sp., Tribonema sp., Spirogyra sp., etc. The source water can include nutrients such as chlorophyll-a, nitrogen, phosphorus, algal mats, and/or macrophytes. In some cases, the total phosphorus (TP) concentration in the source water can be at least about 1 mg/L, at least about 2 mg/L, at least about 3 mg/L, at least about 4 mg/L, and/or at least about 5 mg/L. In some cases, TP can be as high as 5 mg/L, 6 mg/L, 7 mg/L, 8 mg/L, 10 mg/L, 20 mg/L, and/or 50 mg/L. In some cases, the phosphorus (P) can be dissolved and/or in a bioavailable form (e.g., as orthophosphate-P). The total nitrogen (TN) concentration in the source water can be at least about 10 mg/L, at least about 15 mg/L, at least about 19 mg/L, and/or at least about 25 mg/L. In some cases, TN can be as high as 20 mg/L, 22 mg/L, 25 mg/L, 30 mg/L, 40 mg/L, 60 mg/L, and/or 80 mg/L. In some cases, the nitrogen (N) can be dissolved and/or in a bioavailable form (e.g., as ammonia).

The algae in the raceway ponds 120 can consume the nutrients in the source water thereby reducing the concentration of nutrients in the source water. In some cases, the concentration of nutrients can be reduced by at least about 50%, 60%, 70, 80%, and/or 90%. Thus, the concentration of TP and TN in the water can be reduced by as much as about 90% after treatment in the raceway ponds 120 but before treatment in the attached algae flow ways 140, which are further described below. For instance, the concentration of total phosphorus (TP) in the water after algae is cultivated in the raceway ponds can be less than about 1 mg/L, less than about 0.8 mg/L, and/or less than about 0.5 mg/L. The concentration of total nitrogen (TN) in the water after algae is cultivated in the raceway ponds can be less than about 5 mg/L, less than about 4 mg/L, and/or less than about 2 mg/L. Depending on initial nutrient loads in the water source 110, both TP and TN could be reduced by treatment in the raceway ponds 120 to zero or the lower limits of measurement, but typically some level of TN and TP remains, such as greater than about 0.1 mg/L and less than about 0.5 mg/L of TP, and/or greater than about 0.5 mg/L and less than about 2 mg/L of TN prior to treatment in the next stage of the system 100.

In some cases, the system 100 can also include one or more attached algae flow ways 140 (AAFWs). The attached algae flow ways 140 can be positioned downstream of the raceway ponds 120. The algae flow ways 140 can receive the water from the raceway ponds 120. In some cases, algae can be removed from the algae flow ways 140, outputting water with further reduced concentration of nutrients in the water. For example, the algae flow ways 140 can be configured to harvest periphytic algae biomass, including the nutrients being remediated. The concentration of nutrients can be further reduced by at least about 50%, 60%, 70, 80%, and/or 90% relative to the effluent received from the raceway ponds 120. Thus, in the attached algae flow ways 140, the concentration of TP in the water can be further reduced by about 90% or more (relative to the effluent received from the raceway ponds 120). In the attached algae flow ways 140, the concentration of TN in the water can be reduced by as much as about 80% or more (relative to the effluent received from the raceway ponds 120). For instance, the concentration of total phosphorus (TP) in the water after algae is removed from the algae flow ways 140 can be less than about 0.01 mg/L, less than about 0.0075 mg/L, and/or less than about 0.005 mg/L. The concentration of total nitrogen (TN) in the water after algae is removed from the algae flow ways 140 can be less than about 0.1 mg/L, less than about 0.05 mg/L, and/or less than about 0.02 mg/L. Depending on nutrient loads in the water received from the raceway ponds 120, both TP and TN could be reduced by treatment in the raceway ponds 120 to zero or the lower limits of measurement, but TP typically remains greater than about 0.045 mg/L and/or TN typically remains greater than about 0.4 mg/L.

The system 100 can also include a holding tank 150. The holding tank 150 can be positioned downstream of the one or more attached algae flow ways 140. The holding tank 150 can receive at least a portion of the water treated in the algae flow ways 140. The water in the holding tank 150 can be tested to measure the concentration of nutrients. In some cases, the water in the holding tank 150 can be directed to the raceway ponds 120 and/or the algae flow ways 140 for further treatment. In other cases, the water in the holding tank 150 can be returned to water source 110 (e.g., the river, a pond, a stream, etc.) or to another fresh water body. For example, the treated water can be returned directly to the body of fresh water 110 the water was originally received from. The returned water will have a lower concentration of nutrients than when the water entered the system 100. This can beneficially reduce the potential of harmful algal blooms (HABs) in the fresh water body (e.g., the original water source) to which the water is returned.

The system 100 can address surface water remediation and mitigate harmful algal blooms (HABs). The system 100 can be used to implement a method that uses algae to swiftly remove dissolved nutrients from water, significantly reducing nutrient release into surface waters. The system 100 can combine raceway ponds 120 and attached algae flow ways 140, forming an effective method to capture dissolved nutrients before they can contribute to HABs downstream. While raceway ponds 120 and algae flow ways 140 are each individually used to harvest algae, their combination does not significantly increase algae production relative to the algae production of one the two subsystems; however, their combination does significantly reduce nutrient loads in the effluent water.

The treatment approach maximizes nutrient removal, producing a valuable byproduct that can be sold as commercial biofertilizer. The system can also include algae separation systems such as those described in U.S. Pat. No. 12,110,237,, titled ALGAE SEPARATION SYSTEM, the disclosure of which is hereby incorporated herein by reference in its entirety. Such algae separation systems can be employed along the attached algae flow ways to harvest periphytic algae biomass along with their absorbed nutrients. The system 100 can integrate an algae separation system similar to that of the above patent disclosure and algae raceway ponds to achieve efficient nutrient removal across a wide range of phosphorus concentrations, targeting effluent levels as low as about 0.01 mg/L. This dual system can manage seasonal variations in nutrient concentrations, ensuring comprehensive treatment and significant nutrient reduction. Although reference is made to the system 100 being used in combination with other systems, the system 100 can be used without additional systems.

The system 100 harnesses the power of algae to rapidly remove dissolved nutrients from the water. By doing this in a side-stream process, the system 100 can significantly reduce the nutrients being released to surface water. The system 100 can combine raceway algae growth ponds (raceway ponds 120) and attached algae flow ways 140 (AAFWs). The system 100 can remove dissolved nutrients in surface water bodies before they are transported downstream in the water source 110 where they would be able to produce HABs. The system 100 aims to maximize nutrient removal to reduce nutrient inflows into impaired water bodies. At the same time, nutrient-rich harvested algae can serve as a valuable byproduct that can be sold as commercial biofertilizer.

The system 100 can address large variations in P and N concentrations over different seasons. The raceway ponds 120 can remove nutrients when present in high concentrations, and the attached algae flow ways 140 providing further treatment to achieve low levels of nutrients.

The system 100 offers a cost-effective solution for removing nutrients from contaminated waters through a side-stream treatment approach, and can be implemented without chemical application. The system 100 can prevent HABs while simultaneously producing organic fertilizers and biofuels in a circular economic process, resulting in significant cost savings. By harvesting the algae, it becomes directly usable as an organic fertilizer, and upon scale-up, it can undergo anaerobic digestion for further utilization.

In some cases, the system 100 can remove dissolved nutrients (such as phosphorus and nitrogen) from a body of water thereby reducing the load of bioavailable nutrients to downstream bodies of water that contribute to macrophyte impairments in the downstream bodies of water.

In some cases, each of the raceway ponds 120 can have an area of about 100-square-feet. Each of the algae flow ways 140 can also have an area of about 100-square-feet. In cases where the system 100 includes two raceway ponds 120 and two algae flow ways 140, each of which can be considered reactors for absorbing nutrients, and particularly nitrogen and phosphorus, from the water. An example system 100 can thus occupy an area of about 400 square feet. The skilled artisan will appreciate that the system can be scaled up or down.

During operation of the system 100, nutrient-rich water can be pumped to the system 100 for treatment. The water can enter the raceway ponds 120 for initial treatment by planktonic filamentous microalgae to remove N and P. The algae can be harvested with simple screens. The effluent from the raceways ponds 120 can be directed to the attached algae flow ways 140 as a polishing step to further reduce nutrients to very low levels (<0.03 mg/L). The attached algae can be harvested by scraping the flow ways 140. The final treated low-nutrient water can be discharged to the water source 110 or a different body of water, either directly or by way the holding tank 150 in which treated water can be analyzed for nutrient loads and additionally treated if the analysis so indicates. Cultivated algae harvested from the system 100 can be dewatered by settling and draining, air drying (under transparent cover), and/or storing on-site.

The system 100 can include sensors to monitor the operational conditions relevant to algae biomass production and performance of the system 100. The parameters can include flow, temperature, pH, conductivity, dissolved oxygen, turbidity, chlorophyll-a by fluorometry, etc. Parameters such as total and volatile suspended solids, total metals, algae identification and enumeration, chlorophyll-a, dissolved and total organic carbon and inorganic can also be measured.

Algae slurry and/or dewatered algae recovered from the system 100 can be analyzed for nutrient content and used as an organic biofertilizer. The system 100 can be used in various flow and weather conditions.

This system 100 can use filamentous algae (“good algae”) to remove nutrients, N and, principally, P, that encourage HABs to grow. By removing nutrients from source waters, nutrient loads can also be reduced in downstream bodies of water thus reducing the potential for HABs. In some cases, the system 100 can remove at least about 90% P and at least about 80% N from the water that it treats. The system 100 can provide a nature-based solution that harnesses the power of algae to remove nutrients from impaired water bodies. The system 100 can provide a low-cost tool for the removal of legacy nutrients from watersheds.

Algae can reproduce very rapidly, sometimes capable of doubling or tripling their original number in a matter of a day under favorable conditions, and efficiently and rapidly take up nutrients during growth. The system 100 can initially remove at least about 90% of the total P and at least about 80% of the total N at the raceway ponds 120, with a large volume of nutrients continuously removed at a rate greater than any other biological process crops. Large concentrations of N and P can support ˜250 mg/L of algal biomass, based on an 8% N and 2% P content in algae raceway ponds 120, with residual concentrations of these nutrients reduced by about 90% or more, to under 2 mg/L N and less than 0.5 mg/L P. The nutrients can then be further reduced by nearly about 90%, relative to the water released from the raceway ponds 120, by the attached algae flow ways 140.

Algae cultivation for a variety of purposes, including for human consumption, has been practiced commercially for the past 60 years. Algae grow rapidly and can double or triple biomass within 24 hours given optimum growth conditions. Algae require less space to grow and are much cheaper than traditional nutrient reduction methods, such as wetlands and farmland crops. There are only a few commercial processes using the power of algae to remove nutrients from waste and surface waters, and those use expensive technologies for algae cultivation (glass tubes, artificial lights, or complex belt systems, etc.) and harvesting (centrifuges, chemical coagulation, membranes, etc.) The system 100 can beneficially use filamentous algae, allowing for simple cultivation systems (raceway ponds, attached flow ways) and low-cost harvesting of the algal biomass, and therein recovered nutrients, while reducing phosphorus and nitrogen to very low levels.

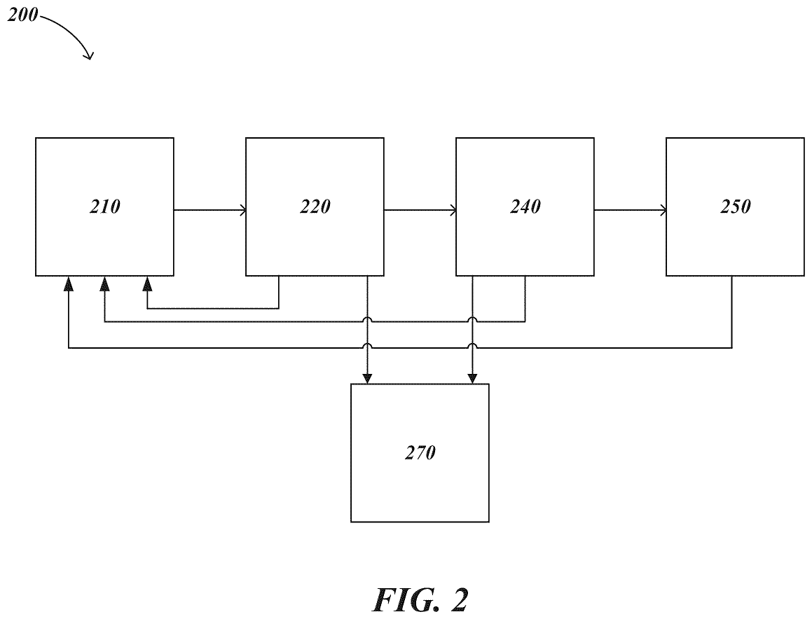

FIG. 2 shows a schematic diagram of a system 200 for algae cultivation and harvesting. The system 200 can be similar or identical to the system 100, with like features represented by like reference numbers incremented by 100. In particular, the system 200 is shown to include a water source 210 feeding one or more raceway ponds 220, which in turn feeds one or more algae flow ways 240, which are in fluid communication with one or more holding tanks 250. The algae grown in the raceway pond(s) 220 and the algae flow ways 240 can be collected and stored or transported for at block 270 for use, e.g., as organic fertilizer or other commercial uses. FIG. 2 is a simplified version of FIG. 1 representing one or more of each subsystem with simple schematic blocks, and illustrating possible flow paths or circulation of water among the blocks by way of arrows.

The terms “comprising,” “including,” “having,” and the like are synonymous and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list.

Although certain embodiments and examples have been described herein, it will be understood by those skilled in the art that many aspects of the systems shown and described in the present disclosure may be differently combined and/or modified to form still further embodiments or acceptable examples. All such modifications and variations are intended to be included herein within the scope of this disclosure. A wide variety of designs and approaches are possible. No feature, structure, or step disclosed herein is essential or indispensable.

For purposes of this disclosure, certain aspects, advantages, and novel features are described herein. It is to be understood that not necessarily all such advantages may be achieved in accordance with any particular embodiment. Thus, for example, those skilled in the art will recognize that the disclosure may be embodied or carried out in a manner that achieves one advantage or a group of advantages as taught herein without necessarily achieving other advantages as may be taught or suggested herein.

Moreover, while illustrative embodiments have been described herein, the scope of any and all embodiments having equivalent elements, modifications, omissions, combinations (e.g., of aspects across various embodiments), adaptations and/or alterations as would be appreciated by those in the art based on the present disclosure. The limitations in the claims are to be interpreted broadly based on the language employed in the claims and not limited to the examples described in the present specification or during the prosecution of the application, which examples are to be construed as non-exclusive. Further, the actions of the disclosed processes and methods may be modified in any manner, including by reordering actions and/or inserting additional actions and/or deleting actions. It is intended, therefore, that the specification and examples be considered as illustrative only, with a true scope and spirit being indicated by the claims and their full scope of equivalents.

Conditional language used herein, such as, among others, “can,” “might,” “may,” “e.g.,” and the like, unless specifically stated otherwise, or otherwise understood within the context as used, is generally intended to convey that some embodiments include, while other embodiments do not include, certain features, elements, and/or states. Thus, such conditional language is not generally intended to imply that features, elements, blocks, and/or states are in any way required for one or more embodiments or that one or more embodiments necessarily include logic for deciding, with or without author input or prompting, whether these features, elements and/or states are included or are to be performed in any particular embodiment.

The ranges disclosed herein also encompass any and all overlap, sub-ranges, and combinations thereof. Language such as “up to,” “at least,” “greater than,” “less than,” “between,” and the like includes the number recited.

1. A method for surface water remediation and prevention and mitigation of harmful algal blooms, comprising:
providing water from a surface water source to a raceway pond;
cultivating algae in the raceway pond to absorb and reduce nutrient concentrations; and
providing water from the raceway pond to an algae flow way connected downstream of the raceway pond; and
cultivating algae in the algae flow way to further reduce nutrient concentrations in the water.

2. The method of claim 1, wherein the raceway pond and the algae flow way are operated as side-stream process for treating water from the surface water source and returning water to the surface water source.

3. The method of claim 1, wherein the method reduces nutrient concentrations in the surface water source to less than about 0.1 mg/L.

4. The method of claim 1, further comprising harvesting algae cultivated in the raceway pond and the algae flow way to produce a commercial biofertilizer.

5. The method of claim 1, further comprising removing algae from the algae flow way to enhance nutrient removal efficiency.

6. The method of claim 1, wherein cultivating algae in the raceway pond reduces a total phosphorus concentration in the water from more than about 4 mg/L to less than about 0.5 mg/L.

7. The method of claim 6, wherein cultivating algae in the algae flow way reduces the total phosphorus concentration to less than about 0.005 mg/L.

8. The method of claim 1, wherein cultivating algae in the raceway pond further reduces a total nitrogen concentration in the water from more than about 19 mg/L to less than about 2 mg/L.

9. The method of claim 8, wherein cultivating algae in the algae flow way further reduces the total nitrogen concentration in the water to less than about 0.02 mg/L.

10. (canceled)

11. (canceled)

12. (canceled)

13. (canceled)

14. (canceled)

15. (canceled)

16. (canceled)

17. (canceled)

18. (canceled)

19. (canceled)

20. (canceled)

21. (canceled)

22. (canceled)

23. (canceled)

24. (canceled)

25. A system for surface water remediation, the system comprising:
a raceway algae growth pond configured to receive source water from a surface body of water and cultivate a first type of algae to absorb and reduce nutrient concentrations in the source water; and
an attached algae flow way positioned downstream of the raceway algae growth pond and configured to receive at least a portion of water treated by the raceway algae growth pond and cultivate a second type of algae to further absorb and reduce nutrient concentrations in the water received from the raceway algae growth pond.

26. The system of claim 25, wherein the raceway algae growth pond is configured to reduce a total phosphorus (TP) concentration of the source water from about 4 mg/L or more to about 0.5 mg/L or less.

27. The system of claim 26, wherein the attached algae flow way is configured to further reduce a total phosphorus (TP) concentration of the water treated by the raceway algae growth pond to about 0.005 mg/L or less in the water treated by the attached algae flow way.

28. The system of claim 25, wherein the raceway algae growth pond is configured to reduce a total nitrogen (TN) concentration of the source water from about 19 mg/L or more to about 2 mg/L or less in the water treated by the raceway algae growth pond.

29. The system of claim 28, wherein the attached algae flow way is configured to further reduce a total phosphorus (TP) concentration of the water treated by the raceway algae growth pond to about 0.02 mg/L or less in the water treated by the attached algae flow way.

30. The system of claim 25, wherein the raceway algae growth pond is configured to reduce the nutrient concentration of the source water by at least about 90%.

31. The system of claim 30, wherein the attached algae flow way is configured to further reduce the nutrient concentration of the water treated by the raceway algae growth pond by at least about 80%.

32. The system of claim 25, wherein the first type of algae comprises planktonic algae.

33. The system of claim 25, wherein the second type of algae comprises periphytic algae.

34. (canceled)

35. (canceled)

36. The system of claim 28, further comprising a holding tank positioned downstream of the attached algae flow way and configured to receive and store at least a portion of the water treated by the raceway algae growth pond and the attached algae flow way.

37. The system of claim 25, further comprising a flow path from the attached algae flow way to the surface body of water.
